(19) United States
(12) Patent Application Publication
(10) Pub. No.: US 2009/0081793 A1
(43) Pub. Date: Mar. 26, 2009

(54) Method for improving acid and low pH tolerance in yeast

(75) Inventors: Danilo Porro, Erba (IT); Laura Dato, Catania (IT); Paola Branduardi, Milano (IT)

Correspondence Address: RATNERPRESTIA, P.O. BOX 1596, WILMINGTON, DE, 19899, US

(73) Assignee: Tate & Lyle Americas, Inc.

(21) Appl. No.: 12/156,567

(22) Filed: Jun. 3, 2008

Related U.S. Application Data
(60) Provisional application No. 60/933,338, filed on Jun. 6, 2007.

(51) Int. Cl. C12N 15/81

(52) U.S. Cl. 435/471

(57) ABSTRACT

A method for increasing tolerance in yeast to organic acids and low pH comprising functionally transforming a yeast with at least one copy of a nucleotide sequence encoding a plasma membrane H⁺-ATPase.

### RELATED APPLICATIONS

This Application claims the benefit of U.S. Application No. 60/933,338, filed on Jun. 6, 2007, which is incorporated herein by reference in its entirety.

### FIELD OF THE INVENTION

The present invention relates generally to the field of increasing tolerance in yeast to organic acids present in culture medium, and to low pH of the medium. More specifically, it relates to increasing H⁺-ATPase levels in yeast used in industrial production.

### BACKGROUND OF THE INVENTION

Yeast are easily grown on an industrial scale, and are frequently employed in the commercial production of compounds such as yeast biomass, organic acids, amino acids, vitamins, polyols, solvents, biofuels, therapeutics, vaccines, proteins, and peptides. However, in industrial processes where yeast are used as a means for production, i.e., fermentation/bioconversion, environmental stresses can lead to reduced or no production of the product, reduced or no productivity, reduced or no yield of the product, or any combination of these problems.

One type of stress, low pH (acidification) of the culture medium is one of the most limiting environmental constraints in yeast fermentation and/or bioconversion. Acidification of the culture medium can occur, for example, if the yeast are engineered to produce industrial products, such as organic acids, that acidify the medium. However, a decrease of the extracellular pH is also commonly observed during yeast fermentation processes.

Direct consequences of lowered pH in the yeast extracellular environment include a decrease in yeast intracellular pH and growth inhibition (Holyoak, et al., Appl. Environ. Microbiol. 62: 3158-3164, 1996; Viegas, et al., Appl. Environ. Microbiol 64: 779-783, 1998). To counteract this effect, yeast rely on a plasma membrane proton pump, an H⁺-ATPase. In Saccharomyces cerevisiae this protein is encoded by the PMA1 gene. The plasma membrane H⁺-ATPase couples ATP hydrolysis to the active transport of protons out of the cell. It is the major plasma membrane protein, and its function is both energetic and regulatory (Michelet and Boutry, Plant Physiol. 108: 1-6, 1995; Goossens et al., M.C.B. 20: 7654-7661, 2000; Morsomme, et al., Biochem. Biophys. Acta 1469: 133-157, 2000; Portillo, Biochim Biophys Acta 1469: 31-42, 2000). The electrochemical proton gradient generated by this enzyme provides the driving force for ion and nutrient transport. Plasma membrane H⁺-ATPase activity correlates with growth rate and stress responses in yeast, and regulation of the protein can occur both transcriptionally and post-translationally.

Two main factors have been shown to control this H⁺-ATPase activity in S. cerevisiae yeast cells: glucose and acid pH. PMA1 transcription can be triggered by glucose metabolism and by cell cycle-dependent regulation. Relatively constant levels of H⁺-ATPase are maintained during yeast growth, because PMA1 is highly expressed and the Pma1 H⁺-ATPase has a relatively long half-life (Mason et al., Biochim. Biophys. Acta 1372: 261-171, 1998; Capieaux, et al., Biochim. Biophys. Acta 1217: 74-80, 1994; Benito, et al., Biochim. Biophys. Acta 1063: 265-268, 1991). Glucose activation has also been observed in other yeast species, e.g., Schizosaccharomyces pombe and Candida albicans (Serrano, FEBS Lett. 156: 11-4, 1983). H⁺-ATPase responses to environmental stresses are thought to be primarily regulated post-translationally. For example, enzyme activity is reduced by removal of glucose and lowered temperature (Mason, et al., Biochim. Biophys. Acta 1372: 261-271, 1998).

Pma1 H⁺-ATPase activity increases in the presence of ethanol (Rosa and Sa-Correia, Appl. Environ. Microbiol. 57: 830-835, 1991); weak organic acids (Viegas and Sa-Correia, J. Gen. Microbiol. 137: 645-651, 1991; Alexandre et al., Microbiol. 142: 469-475, 1996; Holyoak, et al., Appl. Environ. Microbiol. 62: 3158-3164, 1996; Carmelo et al., Biochim. Biophys. Acta 1325: 63-70, 1997; Viegas, et al., Appl. Environ. Microbiol. 64: 779-783, 1998; Macpherson et al., Microbiol. 151: 1995-2003, 2005); supra-optimal temperatures (Viegas et al., Appl. Environ. Microbiol. 61: 1904-1909, 1995); heat shock (Piper et al., Cell Stress Chaperones 2: 12-24, 1997); and deprivation of nitrogen source (Benito, et al., FEBS Lett. 300: 271-274, 1998).

### SUMMARY OF THE INVENTION

The tolerance of a yeast cell to organic acids and/or low pH is increased by functionally transforming a yeast cell with at least one copy of a nucleotide sequence encoding a plasma membrane H⁺-ATPase. Preferably this is accomplished when one or more organic acids is present in the yeast growth medium, when the pH of the growth medium is low, or both.

### BRIEF DESCRIPTION OF THE DRAWINGS

FIG. 1 is a series of graphs showing the effects of PMA1 transformation in S. cerevisiae cells grown in minimal medium with or without added lactic acid, under different conditions of agitation.

FIG. 2 presents graphs showing the effects of PMA1 transformation on the specific activity of Pma1 H⁺-ATPase in S. cerevisiae grown in minimal medium with or without added lactic acid.

FIG. 3 shows an immunoblot of Pma1 H⁺-ATPase protein levels in transformed and control S. cerevisiae cells grown in minimal medium with or without added lactic acid.

FIG. 4 shows histograms displaying the effects of PMA1 transformation on yeast cell viability.

### DETAILED DESCRIPTION OF THE INVENTION

The invention relates to a method of increasing tolerance in yeast to the presence of organic acids in the yeast environment and to low pH in the yeast environment. In the use of yeast for industrial production, the yeast environment is generally a culture or fermentation/bioconversion medium. Organic acids may be exogenously added to the medium, or may be produced by the yeast and accumulate in the medium. Similarly, low pH in the medium may result from yeast metabolism and production, or from the need to employ a low pH medium in an industrial process. For the invention described herein, low pH is a pH of less than or equal to 4.5.

Plasma membrane H⁺-ATPase prevents a deleterious decline in yeast intracellular (cytosolic) pH by pumping protons out of the yeast cell, thus sustaining yeast growth and viability, and consequently, production of a product. To a certain extent, the activity of plasma membrane H⁺-ATPase increases in yeast as the culture medium acidifies.

In one embodiment, the invention provides a method for enhanced expression of a yeast plasma membrane H⁺-ATPase gene, PMA1, by functionally transforming a yeast cell with a nucleotide sequence encoding a plasma membrane H⁺-ATPase to increase organic acid and/or low pH tolerance in yeast. This method is particularly useful for maintaining viability and production in yeast such as yeast biomass, as well as specific intracellular or extracellular components of the yeast biomass, and commercial products such as organic acids, amino acids, vitamins, polyols, solvents, biofuels, therapeutics, vaccines, proteins, and peptides.

A “recombinant” cell or organism is one that contains a nucleic acid sequence that is not naturally occurring in that cell or organism, or one that contains an additional copy or copies of an endogenous nucleic acid sequence, wherein the nucleic acid sequence is introduced into the cell or organism or into an ancestor cell thereof by human action. Introduction of the gene into the cell or organism is known as “transformation” and the recipient organism or cell is said to be “transformed.” Recombinant DNA techniques are well-known to those of ordinary skill in the art, who will also understand how to choose appropriate vectors and promoters for the transformation of yeast strains. (For example, see methods in Sambrook and Russell, Molecular Cloning: A Laboratory Manual, 3rd Edition, Cold Spring Harbor Laboratory Press, 2001).

In one well-known technique, a desired coding region is isolated by first preparing a genomic DNA library or a cDNA library, and second, identifying the coding region in the genomic DNA library or cDNA library, such as by probing the library with a labeled nucleotide probe that is at least partially homologous with the coding region, determining whether expression of the coding region imparts a detectable phenotype to a library microorganism comprising the coding region, or amplifying the desired sequence by PCR. Other techniques for isolating the coding region may also be used.

Methods for preparing recombinant polynucleotides and transferring them into a host organism are well-known to those of ordinary skill in the art. One such method is described in detail in Example 2. In general, the desired coding region is incorporated into the recipient organism in such a manner that the encoded protein is produced by the organism in functional form, a procedure known as “functional transformation.” That is, the coding region is inserted into an appropriate vector and operably linked to an appropriate promoter on the vector. If necessary, codons in the coding region may be altered, for example, to create compatibility with codon usage in the target organism, to change coding sequences that can impair transcription or translation of the coding region or stability of the transcripts, or to add or remove sequences encoding signal peptides that direct the generated protein to a specific location in or outside the cell, e.g., for secretion of the protein. Any type of vector, e.g., integrative, chromosomal, or episomal, may be used. The vector may be a plasmid, cosmid, yeast artificial chromosome, virus, or any other vector appropriate for the target organism. The vector may comprise other genetic elements, such as an origin of replication to allow the vector to be passed on to progeny cells of the host carrying the vector, sequences that facilitate integration into the host genome, restriction endonuclease sites, etc. Any promoter active in the selected organism, e.g., homologous, heterologous, constitutive, inducible, or repressible may be used.

An “appropriate” vector or promoter is one that is compatible with the selected organism and will allow that organism to generate a functional protein. The recombinant organism thus transformed is referred to herein as being “functionally transformed.”

The recombinant organisms of the invention can be transformed by any method allowing a foreign DNA to be introduced into a cell, for example, chemical transformation, electroporation, conjugation, fusion of protoplasts or any other known technique (Spencer J. F. et al., Journal of Basic Microbiology 28: 321-333, 1988). A number of protocols are known for transforming yeast. Transformation can be carried out by treating the whole cells in the presence of lithium acetate and of polyethylene glycol according to Ito H. et al. (J. Bacteriol. 153: 163, 1983), or in the presence of ethylene glycol and dimethyl sulphoxyde according to Durrens P. et al. (Curr. Genet. 18: 7, 1990). An alternative protocol has also been described in EP 361991. Electroporation can be carried out according to Becker D. M. and Guarente L. (Methods in Enzymology 194:18, 1991). The use of non-bacterial integrative vectors may be preferred when the yeast biomass is used at the end of the fermentation process as stock fodder or for other breeding, agricultural or alimentary purposes.

The transformed yeast cell is propagated in an appropriate culture medium. Culturing techniques and specialized media are well known in the art. For industrial production, the organism is preferably cultured either in free suspension or immobilized in an appropriate medium in a fermentation vessel.

In one embodiment, the transformed yeast are also engineered for industrial production of a product. Such products may include organic acids, amino acids, vitamins, polyols, solvents, biofuels, therapeutics, vaccines, proteins, and peptides. Organic acids produced by the transformed yeast may include lactic, citric, malic, fumeric, succinic, ascorbic, pyruvic, itaconic, malonic, acetic, benzoic, malic, and sorbic acids.

Although S. cerevisiae is commonly used for industrial processes, other types of yeast are also appropriate for this invention. These include, but are not limited to, the genera, Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, Schwanniomyces, and Torulaspora.

SEQ ID NO:1 is the sequence of a plasma membrane H⁺-ATPase (PMA1) from S. cerevesiae. In one embodiment of the invention, the nucleotide sequence encoding a plasma membrane H⁺-ATPase is SEQ ID NO:1. However, PMA1 sequences from other species (either from yeast or other organisms) may also be used to practice the invention, which is not limited to transformation with SEQ ID NO:1. See, for example, de Kerchove d'Exaerde, et al., J. Biol. Chem 270: 23828-23837, 1995; Gorgojo, et al., Biochim. Biophys. Acta 1509: 103-110, 2000; Luo et al., Plant Physiol. 119: 627-634, 1999; Struck, et al., Mol. Plant Microbe Interact. 11: 458-465, 1998 A simple growth assay, comparing growth of the transformed yeast in minimal medium and growth in minimal medium with an organic acid added and/or at low pH, such as that described in Example 1, may be used to determine whether a PMA1 sequence from a particular species will increase tolerance of a particular yeast species to organic acids and/or low pH.

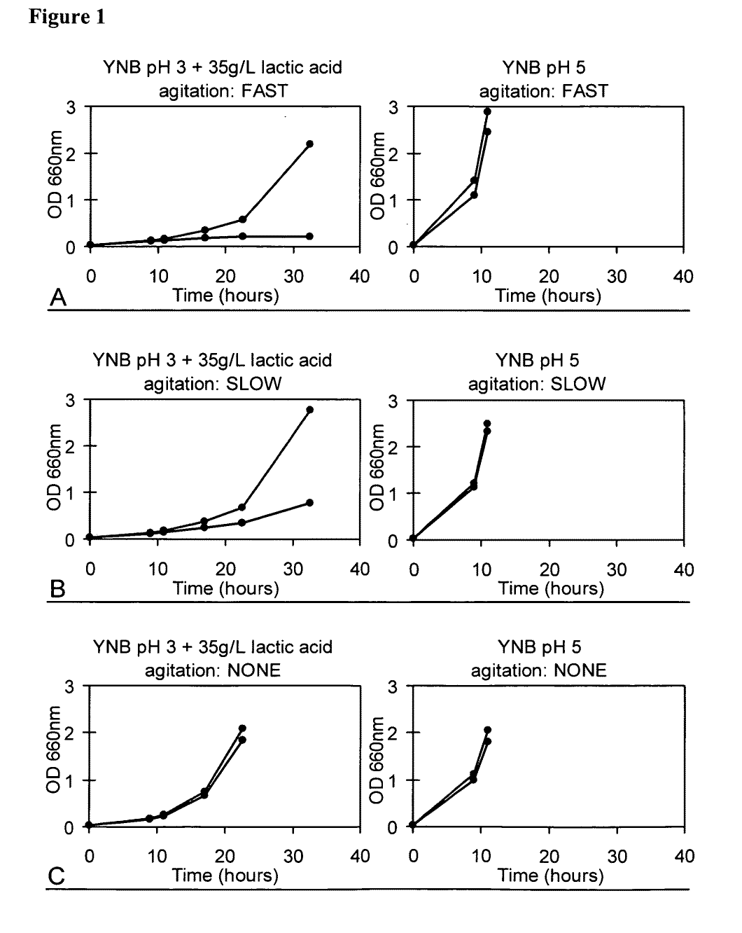

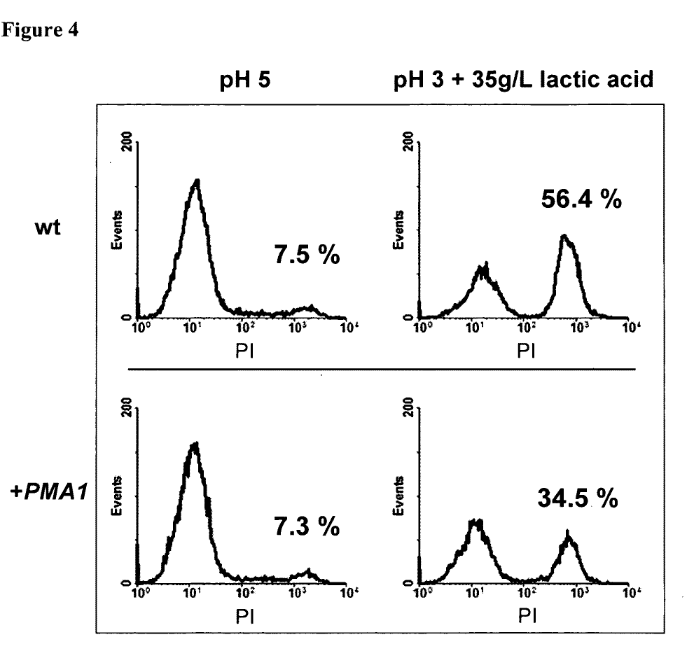

Supplementary expression of an endogenous Pma1 H⁺-ATPase activity renders the transformed yeast capable of growth and/or production, even in environmental conditions that are limiting for yeast cells that are not functionally transformed with PMA1, e.g., presence of organic acids in the medium and low pH medium, as shown in FIGS. 1-3 and described in Examples 1-5. In addition, supplementary expression of endogenous Pma1 H⁺-ATPase activity enhances yeast viability under limiting conditions, as shown in FIG. 4 and described in Example 6.

Generation of reactive oxygen species (ROS) are related to many stresses (Apel and Hirt, Ann. Rev. Plant Biol. 55: 373-399, 2004; Temple, et al., Trends Cell Biol. 15: 319-326, 2005; Riter, et al., Antioxid. Redox Signal 9: 49-89, 2007. Generally speaking, stresses may have cellular (internal) origins, environmental (external) origins, or both (Salmon, et al., Nucl. Acids Res. 32: 3712-3723, 2004). Classic examples of internally-originating stresses include protein and metabolite overproduction (Xiao, et al., Appl. Microbiol. Biotechnol. 72: 837-844, 2006. Examples of externally-originating stresses include, among others, high osmolarity, high salinity, oxidative stress, high or low temperature, high or low pH values, presence of organic acids, presence of toxic compounds, and macro- and micro-nutrient starvation. Regardless of their origin, stresses on microorganisms can have various deleterious effects, including lower metabolic activity, lower growth rate, lower productivity and/or lower viability.

Agitation of yeast cultures oxygenates the cultures, which can lead to severe oxidative stress from ROS when organic acids are present in the medium (Piper, Free Radical Biol. Med. 27: 1219-1227, 1999). However, as shown in FIG. 1, yeast transformed with PMA1 continue to grow in the presence of organic acids and low pH, even when the culture is agitated at high speed, while growth is inhibited in nontransformed yeast under these conditions.

### EXAMPLES

1. Growth Conditions

The following S. cerevisiae strains were utilized: CEN.PK 102-5B (Mat a, his 3Δ1) [pYX012] [pYX042] and CEN.PK 102-5B (Mat a, his 3Δ1) [pYX012] [pYX042ScPMA1], both derived from the parental strain CEN.PK 102-5B (Mat a, ura3-52, his 3Δ1, leu2-3,112).

All experiments were performed in YNB minimal medium (Yeast Nitrogen Base—DIFCO) containing 2% (w/v) glucose and 50 mg/L histidine (for strain auxotrophy), supplemented or not with the indicated concentrations of lactic acid. The initial pH of the medium without added organic acid was approximately 5.0. When an organic acid was added to the medium, the initial pH of the medium was adjusted to 3.0 by adding HCl or NaOH.

2. ScPMA1 Amplification, Plasmid Construction, and Yeast Transformation

The S. cerevisiae strain CEN.PK was transformed with an integrative plasmid bearing the endogenous gene PMA1, encoding the plasma membrane Pma1 H⁺-ATPase, posed under the control of the glycolytic TPI promoter (and with the same, but empty, plasmid as a control). Independent clones (at least three for each transformation) were grown in minimal medium (pH 4.8-5) and in minimal medium added with lactic acid (at different concentrations, from 20 to 35 g/l) at pH3 in microtiter plates at 30° C. under vigorous shaking or with no agitation.

The ScPMA1 gene was amplified from the CEN.PK genomic DNA by PCR. For the amplification, the following primers were used:
ScPMA1_fw: 5′-ATC AT ATG ACT GAT ACA TCA TCC TC-3′ (SEQ. ID NO: 2);
ScPMA1_REV: 5′-ACA GGA TTA GGT TTC CTT TTC GTG-3′ (SEQ. ID NO: 3).

Amplification was obtained by using the Polymerase Pwo DNA (Roche, Mannheim, Germany). The annealing temperature was 57° C. and the elongation time was 2 min. The resulting DNA fragment was sub-cloned into the shuttle plasmid vector pSTBlue-1, using the Perfectly Blunt Cloning Kit (Novagen, Darmstadt, Germany), and was subsequently sequenced to verify the correct amplification. The plasmid was then digested with ApaI-MluI to release the ScPMA1 gene fragment, which was then was ligated into the pYX042(-ATG) expression plasmid at the ApaI-MluI site. The pYX042(-ATG) plasmid was prepared from the pYX042 integrative expression vector (R&D Systems, Wiesbaden, Germany) by digesting with EcoRI and BamHI, blunt-ending, and re-ligating.

For yeast transformation, the integrative plasmids pYX042 and pYX042-ScPMA1 were linearized at the HpaI and NarI sites, respectively, and used for leu2 complementation. Yeast transformation was performed using the lithium acetate/ssDNA method, as described by Gietz and Woods, Methods Mol. Biol. 313: 107-120, 2006.

3. Effect of PMA1 Transformation on Growth Rate in the Presence of Lactic Acid

To determine the effects of PMA1 transformation on yeast growth in the presence of organic acids, transformed and control yeast were grown in minimal medium to which lactic acid (35 g/L) was added, pH 3.0. Cultures were agitated at fast or slower speeds, or grown without agitation. Yeast cells were pre-cultured in liquid glucose minimal medium and then re-inoculated in the indicated media. Cells were all inoculated at time 0 at OD (660 nm) 0.03. Growth was allowed at 30° C. in shake flasks with or without agitation in a New Brunswick Scientific, Innova 40 shaking incubator. “Slow agitation” was obtained by shaking at 155 rpm, “fast agitation” at 280 rpm.

Growth rates are shown in FIG. 1. Graphs in the left panel represent yeast in minimal medium to which lactic acid was added. Graphs in the right panel represent yeast in minimal medium. Black circles: CEN.PK cells transformed with the integrative empty plasmid (control). Open circles: CEN.PK cells transformed with an integrative plasmid bearing a ScPMA1 copy under the control of the ScTPI promoter. (A) Fast agitation. (B) Slow agitation. (C) No agitation. Transformed yeast exhibited higher growth rates than control yeast in medium containing lactic acid and low pH, and this effect was more pronounced when the yeast were incubated with agitation. No difference in growth rates was seen between transformed and control yeast in minimal medium without lactic acid, demonstrating that PMA1 transformation has no detectable effect on growth rate in normal medium.

4. Effect of PMA1 Transformation on Specific Activity of Pma1 H⁺-ATPase

This method is based on the measurement of inorganic phosphate released by ATPase activity and is a modification of the assay described by Serrano, FEBS Lett. 156: 11-14. 1988. All yeast cultures were inoculated at OD (660 nm) 0.03, grown under agitation, and collected when they reached the mid-log phase (about OD 2.0). Assays were performed on crude membrane samples, obtained by differential centrifugation of yeast cell homogenates as described in Serrano (1988), and normalized for total protein content. Protein concentrations of the samples were determined by the dye-binding method of Bradford, using BSA as standard.

For each sample, 1 ml of incubation mix was prepared in 100 mM MES (2-morpholinoethanesulfonic acid) buffered to pH 6.0 with TRIS, containing 5 mM MgCl₂, 50 mM KNO₃, 5 mM NaN₃, 0.2 mM ammonium molybdate, and a volume of membranes corresponding to 25 μg of proteins. The reaction was initiated by adding 60 μl of ATP (0.1 M). After 30 min of incubation at 30° C., the reaction was stopped by adding 2 ml of a stop solution containing 2% (v/v) H₂SO₄, 0.5% SDS and 0.5% ammonium molybdate. Color was developed by adding 20 μl of 10% ascorbic acid. The absorbance at 750 nm was determined after waiting at least 10 minutes after addition of ascorbic acid.

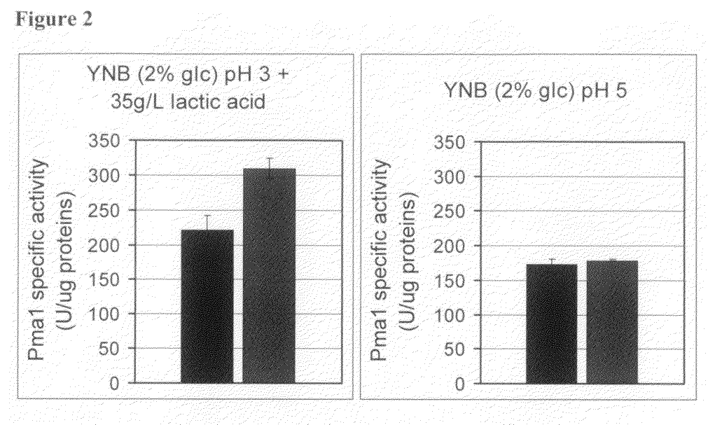

The specific activity of Pma1 H⁺-ATPase is expressed as units per milligram of total protein in the sample. A unit of activity is defined as the amount of enzyme that catalyzes the hydrolysis of 1 pmol of ATP per minute under the assay conditions. Results are shown in FIG. 2, which shows that Pma1 H⁺-ATPase specific activity is generally higher when lactic acid is present in the medium (left panel) than under permissive conditions (right panel) in all strains. This result is in agreement with previous reports from the literature.

In the PMA1 overexpressing cells (grey, CEN.PK cells transformed with an integrative plasmid bearing a ScPMA1 copy under the control of the ScTPI promoter ) the specific activity of Pma1 H⁺-ATPase is higher than in the control strain (black, CEN.PK cells transformed with the integrative empty plasmid). This is clearly evident for yeast grown in conditions of stress (left panel), but the difference is still present, although less pronounced, for samples grown in permissive conditions (right panel). These data indicate that the observed advantage in growth rate in transformed S. cerevisiae cells grown in minimal medium under stress correlates with increased Pma1 H⁺-ATPase specific activity.

5. Immuno-Detection of Pma1 H⁺-ATPase Levels

For immuno-detection of the Pma1 H⁺-ATPase in the crude membrane extracts, the samples were separated by SDS-PAGE (acrylamide concentration: 8%), and blotted on a nitrocellulose membrane for 2.5 hours at 250 mAmps. The membranes were subsequently immuno-labeled with the primary anti-Pma1 antibody (mouse monoclonal, Abcam ab4645 (Cambridge, U.K.)), diluted 1:1500, during a 2 h incubation at room temperature. The secondary anti-mouse antibody (Amersham NA931V (GE Healthcare Bio-Science, Piscataway, N.J., U.S.A.) was used (diluted 1:10,000) for detection and immuno-reactive protein bands were developed using the Super Signal West Pico Western blotting system (Pierce, Rockford, Ill., U.S.A) according to the manufacturer's instruction.

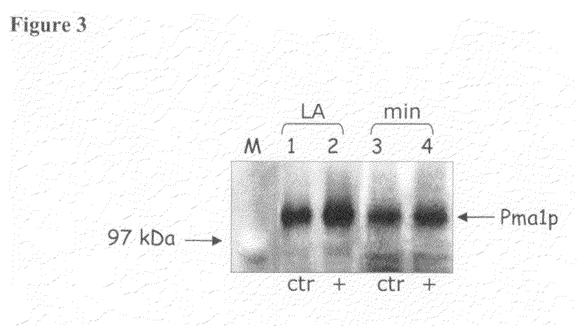

These results are shown in FIG. 3. All cells were inoculated at OD (660 nm) 0.03, grown under agitation and collected when they reached the mid-log phase (about OD 2). Total protein (7.5 ug for lanes 1 and 2, 15 ug for lanes 3 and 4) was loaded for each sample (M=markers). Lanes 1 and 3: CEN.PK cells transformed with the integrative empty plasmid (control). Lanes 2 and 4: CEN.PK cells transformed with an integrative plasmid bearing a SCPMA1 copy under the control of the ScTPI promoter (positive).

Pma1 H⁺-ATPase levels are somewhat greater in samples derived from cells grown under limiting conditions (added lactic acid, lanes 1 and 2) compared with permissive conditions (no added lactic acid, lanes 3 and 4).

In addition, the PMA1-transformed yeast (lanes 2 and 4), express somewhat greater amounts of Pma1 H⁺-ATPase than the control yeast in limiting, but not permissive, medium. Accordingly, these data correlate with the specific activity data shown in FIG. 2.

6. Effect of PMA1 Transformation on Cell Viability

A cytofluorimetric analysis of S. cerevisiae cells grown in minimal medium with or without added lactic acid was conducted to determine the effects of PMA1 transformation on yeast cell viability. Yeast cells were grown as described in Example 3. All cultures were agitated at 155 rpm. Yeast cells were harvested, washed in phosphate buffered saline (PBS), and resuspended in 0.46 mM propidium iodide (PI). PI-labelled cells were sonicated and analysed using a CELL LAB QUANTA™ SC flow cytometer (Beckman Coulter, Fullerton, Calif., USA) equipped with a diode laser (exitation wavelength 488 nm). The fluorescence emission was measured through a 670 nm long pass filter (FL3 parameter). A total of 20,000 events was recorded for each sample.

Results are shown in FIG. 4. Histograms in the left panel represent yeast grown in minimal medium. Histograms in the right panel represent yeast grown in minimal medium containing 35 g/L lactic acid, pH 3.0. Upper panels: CEN.PK cells transformed with the integrative empty plasmid (control). Lower panels: CEN.PK cells transformed with an integrative plasmid bearing a ScPMA1 copy under the control of the ScTPI promoter. For each histogram, the peak on the right represents the frequency of events with a high signal for PI, corresponding to damaged and/or dead cells. Addition of lactic acid to the medium greatly increased cell damage and death in control and transformed yeast cultures. However, transformation with PMA1 partially protected the viability of yeast grown in medium containing lactic acid (56% nonviable cells for control yeast vs. 34% nonviable cells for transformed yeast).

Although the invention is illustrated and described herein with reference to specific embodiments, the invention is not intended to be limited to the details shown. Rather, various modifications may be made in the details within the scope and range of equivalents of the claims and without departing from the invention.

What is claimed is:

1. A method for increasing the tolerance of a yeast cell to organic acids comprising the step of functionally transforming a yeast cell with a nucleotide sequence encoding a plasma membrane H⁺-ATPase.

2. The method of claim 1 further comprising the step of culturing the transformed yeast in a growth medium under conditions wherein one or more organic acids becomes present in the growth medium.

3. The method of claim 1, wherein the yeast is selected from the group of yeast genera consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, Schwanniomyces, and Torulaspora.

4. The method of claim 2, wherein the organic acid that becomes present in the growth medium is selected from the group of acids consisting of lactic, citric, fumeric, succinic, ascorbic, pyruvic, itaconic, malonic, acetic, benzoic, malic, and sorbic acids.

5. The method of claim 2, wherein the organic acid that becomes present in the growth medium is added to the medium exogenously.

6. The method of claim 2, wherein the organic acid that becomes present in the growth medium is generated by a yeast cell.

7. The method of claim 1, wherein the nucleotide sequence encoding a plasma membrane H⁺-ATPase consists of SEQ ID NO:1.

8. A method for increasing tolerance of yeast to low pH comprising the step of functionally transforming a yeast with a nucleotide sequence encoding a plasma membrane H⁺-ATPase.

9. The method of claim 8 further comprising the steps of culturing the transformed yeast in a growth medium, wherein the pH of the growth medium is or becomes less than or equal to pH 4.5.

10. The method of claim 8, wherein the yeast is selected from the group of yeast genera consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, Schwanniomyces, and Torulaspora.

11. The method of claim 8, wherein the nucleotide sequence encoding a plasma membrane H⁺-ATPase consists of SEQ ID NO:1.

12. A method for increasing productivity of a yeast cell engineered for industrial production comprising the step of functionally transforming a yeast cell engineered for industrial production with a nucleotide sequence encoding a plasma membrane H⁺-ATPase, wherein the transformed yeast has increased tolerance to organic acids, to low pH, or to both organic acids and low pH.


SEQUENCE LISTING (3 sequences)

SEQ ID NO: 1 - DNA, 2757 bp, Saccharomyces cerevisiae
atgactgatacatcatcctcttcatcatcctcttcagcatcttctgtttcagctcatcagccaactcaagaaaagcctgctaagacttacgatgacgctgcatctgaatcttctgacgatgacgatatcgatgctttaatcgaagaactacaatctaatcacggtgtcgacgacgaagacagtgataacgatggtccagttgccgccggtgaagctagaccagttccagaagaatatttacaaactgacccatcttacggtttaacttccgatgaagttttgaaaagaagaaagaagtacggtttgaatcaaatggctgacgaaaaagaatctttagtcgttaagttcgttatgtttttcgtcggtccaattcaattcgttatggaagccgctgctattttggctgccggtttgtccgattgggtcgatttcggtgttatctgtggtttgttaatgttaaacgctggtgttggtttcgttcaagaattccaagctggttctattgtcgatgaattgaaaaagactttggctaacactgctgttgttatcagagacggtcaattggttgaaattccagccaacgaagtcgtcccaggtgatattttgcaattggaagatggtactgttatcccaactgatggtcgtattgtcactgaagactgtttcttgcaaatcgatcaatctgctattactggtgaatctttggctgtcgacaaacattacggtgaccaaactttctcttcttccactgttaagagaggtgaaggtttcatggttgttaccgctaccggtgacaacactttcgttggtagagctgctgctttggttaacaaagccgctggtggtcaaggtcatttcactgaagttttgaacggtattggtattatcttattggttttggtcattgccactttgttgttggtctggactgcttgtttctacagaaccaacggtattgttagaatcttgagatacactctaggtattactattattggtgtcccagtcggtttgccagctgtcgttaccaccactatggccgtcggtgctgcttacttggctaagaaacaagccattgttcaaaagttgtctgctattgaatccttggctggtgtcgaaatcttgtgttccgacaaaaccggtactttgaccaagaacaagttgtccttgcacgaaccatacactgttgaaggtgtttctccagacgacttgatgttgactgcttgtttggctgcttccagaaagaagaagggtttggatgctattgataaggctttcttgaagtctttgaagcaatatccaaaggctaaggacgctttgaccaagtacaaggttttggaattccatccatttgaccctgtctccaagaaggttactgccgttgtcgaatctccagaaggtgaaagaattgtttgtgttaagggtgctccattattcgtcttgaagactgtcgaagaagatcacccaatcccagaagatgtccatgaaaactacgaaaacaaggttgctgaattggcttctagagggttccgtgctttaggtgttgctagaaagagaggtgaaggtcactgggaaatcttgggtgttatgccatgtatggatccaccaagagacgatactgctcaaactgttagcgaagctagacacttgggtctaagagttaagatgttaactggtgatgctgttggtattgctaaggaaacttgtagacaattgggtttgggtaccaacatttacaacgctgaaagattaggtctaggtggcggtggtgacatgccaggttctgaattagctgattttgttgaaaatgccgatggtttcgctgaagttttcccacaacataaatacagagttgttgaaatcttgcaaaacagaggttacttggttgctatgactggtgatggtgttaacgatgctccatctttgaagaaggctgatactggtattgctgtcgaaggtgctactgatgctgccagatctgctgctgatattgttttcttggctcctggtctatctgctattattgatgctttgaagacctccagacaaatttttcacagaatgtactcttacgttgtttaccgtattgctttgtctctacatttggaaatcttcttgggtctatggattgctattttggataactctttggacattgatttgattgttttcatcgctattttcgctgatgttgctactttggctattgcttacgataatgctccttactctccaaagcccgttaaatggaacctaccaagattatggggtatgtctattattttgggcatagttttagctataggttcttggattaccttgactactatgttcttaccaaagggtggtattatccaaaacttcggtgctatgaacggtattatgttcttgcaaatttccttgactgaaaactggttgattttcattaccagagctgctggtccattctggtcttctatcccatcctggcaattggctggtgccgtcttcgctgtcgacatcatcgctaccatgtttaccttattcggttggtggtctgaaaactggactgatattgttactgtcgtccgtgtctggatctggtctatcggtatcttctgtgttttgggtggtttctactacgaaatgtccacttctgaagcctttgacagattgatgaacggtaagccaatgaaggaaaagaagtctaccagaagtgtcgaagacttcatggctgctatgcaaagagtctctactcaacacgaaaaggaaacctaa

SEQ ID NO: 2 - DNA, 26 bp, Artificial Sequence
  Primer
atcaatatgactgatacatcatcctc

SEQ ID NO: 3 - DNA, 24 bp, Artificial Sequence
  Primer
acaggattaggtttccttttcgtg